(19) United States
(12) Patent Application Publication
(10) Pub. No.: US 2024/0010962 A1
(43) Pub. Date: Jan. 11, 2024

(54) MICROFLUIDIC CELL CULTURE DEVICE AND METHOD FOR CELL CULTIVATION

(71) Applicant: Finnadvance Oy, Oulu (FI)

(72) Inventors: Prateek SINGH, Oulu (FI); Hoang NGUYEN, Oulu (FI); Tuomas NURMI, Oulu (FI)

(73) Assignee: Finnadvance Oy, Oulu (FI)

(21) Appl. No.: 18/037,489

(22) PCT Filed: Sep. 6, 2022
(86) PCT No.: PCT/FI2022/050587

§ 371 Date: May 17, 2023

(30) Foreign Application Priority Data
Sep. 9, 2021 (FI) 20215947

(51) Int. Cl. C12M 3/06; C12M 1/42; C12M 1/12; C12M 1/00

(52) U.S. Cl. CPC C12M 23/16; C12M 35/04; C12M 25/02; C12M 23/26

(57) ABSTRACT

A microfluidic cell culture device for cell cultivation includes two or more media reservoirs, a first microfluidic chamber having a non-planar surface, a second microfluidic chamber being a pressure chamber and a flexible non-porous membrane that separates the first microfluidic chamber and the second microfluidic chamber. The flexible non-porous membrane is opposite to the non-planar surface of the first microfluidic chamber. One or more microfluidic channels connect the first microfluidic chamber to the two or more media reservoirs. Disclosed also is a method of cell cultivation using the aforementioned microfluidic cell culture device.

### TECHNICAL FIELD

The present disclosure relates generally to cell culture techniques, and more specifically to microfluidic cell culture device for cell cultivation. Moreover, the present disclosure is concerned with methods of cell cultivation.

### BACKGROUND

The cells have an inherent property to grow and differentiate in-vivo. However, the cells may also be grown in-vitro to simulate the in-vivo processes associated with an organism. Notably, in-vitro cell culture assays have been an important means for evaluating various cellular interactions, cellular wounding and healing mechanism, and so forth. However, various cell models, for example the nervous system models from cells such as brain, spinal cord, peripheral nerve and neuromuscular junction, are difficult to be replicated or studied using the in-vitro cell culture assays, mostly, as such assays fail to account for mufti-level cellular interactions in their natural setting. Therefore, the nervous system models could be studied using either a membrane model or a channel model.

Membrane models typically use a porous membrane as a scaffold for cell attachment and/or supplying cells with a nutrient media. Normally, microfluidic devices comprising the porous membranes may be employed to replicate and study various cell models, such as vascular models. However, such microfluidic devices are not optimized for brain research, i.e. nervous system models, for example determining an impact of various pressures on the nerve cells. Typically, the porous membranes in such microfluidic devices fail to handle the pressure (liquid pressure through the culture chambers or mechanical pressure onto the membrane) exerted on it to make for a reliable device. Moreover, the porous membranes of the microfluidic devices are often rigid and fragile and are susceptible to being torn, making for an unreliable device. Furthermore, porous membranes also have a disadvantage of being poor at guiding neuronal axon growth and hence can't be used to model nerve damage outside of grey matter accurately.

Recently, microfluidic pressing devices have been produced to solve problems associated with the conventional microfluidic devices. Such microfluidic pressing devices employ a non-porous membrane to separate the microfluidic pressing device into 2 parts, and press against a cell culture grown on a substrate arranged at a suitable location in the microfluidic pressing device. However, existing microfluidic pressing devices are also not optimized for brain research, i.e. nervous system models, as the non-porous membranes cause the difference in chamber pressures to be too high at physiologically relevant levels when trying to mimic brain trauma.

The channel models are more suitable for housing a nervous system cell culture, and aim at forming nerve axons along a certain channel, tube, gel, and the like, through or into which a nerve cell can grow. It will be appreciated that neurons need a physical guide for axon growth. Existing microfluidic channel-based devices are designed to house the nervous system cell culture in channels, and apply pressure to the nervous system cell culture for causing a damage or haemorrhage thereto. However, most of such channels are narrow and the fabrication material thereof is either plastic or polysilicone, therefore, it is hard to exert pressure on such channels in a controlled manner using air or liquid pressure. Thereby, making such microfluidic channel-based devices impractical for studying brain damage, primarily due to low applying pressures.

Currently, mouse models are used to study the nervous system models. Such mouse models typically involve hitting the mouse cranium with a standard force, thus causing brain damage or haemorrhage, and eventually sacrificing the mouse for data collection. However, animal models are not a very efficient in replicating human nervous system models, and moreover, are ethically problematic. Moreover, mice also have high costs associated with purpose-built facilities, trained technicians and long waiting times as mice reproduce and grow in a space of months only to yield one datapoint per mouse. Furthermore, mouse models are associated with requirement of frequent human intervention, thus rendering the conventional techniques inefficient.

Therefore, considering the foregoing discussion, there exists a need to overcome drawbacks associated with conventional techniques for cell cultivation, especially for the study of nerve tissue damage.

### SUMMARY

The present disclosure seeks to provide a microfluidic cell culture device for cell cultivation. The present disclosure also seeks to provide a method of cell cultivation. The present disclosure seeks to provide a solution to the existing problem of in-vitro cell cultivation and understanding the various cellular interactions and/or processes. An aim of the present disclosure is to provide a solution that overcomes at least partially the problems encountered in prior art, and provides an efficient and reliable design for the microfluidic cell culture device that achieves higher quality of cells defined by an optimal growth of the cells for the purposes of analysing wounding and healing in cells, specifically brain cell, or testing potential substances, such as for example drugs, therapeutics, toxins, pollutants, and so forth, thereon.

In one aspect, an embodiment of the present disclosure provides a microfluidic cell culture device for cell cultivation, the microfluidic cell culture device comprising
two or more media reservoirs;
a first microfluidic chamber having a non-planar surface;
a second microfluidic chamber being a pressure chamber,
a flexible non-porous membrane that separates the first microfluidic chamber and the second microfluidic chamber, wherein the flexible non-porous membrane being opposite to the non-planar surface of the first microfluidic chamber; and
one or more microfluidic channels connecting the first microfluidic chamber to the two or more media reservoirs.

In another aspect, an embodiment of the present disclosure provides a method of cell cultivation using a microfluidic cell culture device, the microfluidic cell culture device comprising:
two or more media reservoirs;
a first microfluidic chamber having a non-planar surface;
a second microfluidic chamber being a pressure chamber,
a flexible non-porous membrane that separates the first microfluidic chamber and the second microfluidic chamber, wherein the flexible non-porous membrane being opposite to the non-planar surface of the first microfluidic chamber; and
one or more microfluidic channels connecting the first microfluidic chamber to the two or more media reservoirs, wherein the method comprises cultivating a biological material on a non-planar surface of the first microfluidic chamber.

Embodiments of the present disclosure substantially eliminate or at least partially address the aforementioned problems in the prior art and enable effective supply of media in the microfluidic cell culture device and improved removal of the used culture media for optimal growth of cells.

Beneficially, the microfluidic cell culture device design is suitable to model the human nervous system in order to study an increase in the fluid pressure of the brain by changing the fluid pressure in the first microfluidic chamber, thus providing a much more complex, tuneable, and repeatable systems to model different types of brain injuries.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to scale. Wherever possible, like elements have been indicated by identical numbers.

Embodiments of the present disclosure will now be described, by way of example only, with reference to the following diagrams wherein:

FIGS. 1 and 2 are respectively a perspective view and an expanded view of a microfluidic cell culture device for cell cultivation, in accordance with an embodiment of the present disclosure; and

FIG. 3 is a flowchart of steps of a method for cell cultivation, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible.

In one aspect, an embodiment of the present disclosure provides a microfluidic cell culture device for cell cultivation, the microfluidic cell culture device comprising
two or more media reservoirs;
a first microfluidic chamber having a non-planar surface;
a second microfluidic chamber being a pressure chamber,
a flexible non-porous membrane that separates the first microfluidic chamber and the second microfluidic chamber, wherein the flexible non-porous membrane being opposite to the non-planar surface of the first microfluidic chamber; and
one or more microfluidic channels connecting the first microfluidic chamber to the two or more media reservoirs.

In another aspect, an embodiment of the present disclosure provides a method of cell cultivation using a microfluidic cell culture device, the method comprising
two or more media reservoirs;
a first microfluidic chamber having a non-planar surface;
a second microfluidic chamber being a pressure chamber,
a flexible non-porous membrane that separates the first microfluidic chamber and the second microfluidic chamber, wherein the flexible non-porous membrane being opposite to the non-planar surface of the first microfluidic chamber; and
one or more microfluidic channels connecting the first microfluidic chamber to the two or more media reservoirs, wherein the method comprises cultivating a biological material on a non-planar surface of the first microfluidic chamber.

The present disclosure provides the aforementioned microfluidic cell culture device. The microfluidic cell culture device has microfluidic channels for nervous system cell cultivation and providing guidance for growing cells to position in a desired location and mimic nervous system models. The microfluidic cell culture device allows culturing of cells or any cell containing bodies, such as organoid, spheroids, pieces of tissue, and so forth. Moreover, the microfluidic cell culture device is designed to continuously provide fresh cells (namely, biological sample), nutrient feed and various analytes for an optimal growth of the cells and testing purposes. Furthermore, the microfluidic cell culture device provides efficient production of cells upon growth by effectively mimicking fluid flow in human tissues and organs. The device of the present disclosure allows to study brain organoids by adding sphere like organoids into the device, trapping them in the centre of the device, and applying pressure thereto in order to simulate brain damage, such as a broken skull would cause. Moreover, the microfluidic cell culture device is an overall energy-, time- and cost-efficient solution for studying wounding and healing of cells in real-time. Beneficially, the pressure applied is tuneable, for example in terms of intensity or application time (ranging from milliseconds to minutes) based on the cell type or the experiment type for studying different types of brain injuries. Additionally, beneficially, the microfluidic cell culture device addresses the problem associated with the conventional membrane-based microfluidic pressing devices as well as the channel-based microfluidic devices with regards to the inability thereof for brain research. Additionally, the microfluidic cell culture device (and method of using the same) enables essaying brain damage at high throughput with human cells and avoiding the use of animal models in the field of pre-clinical brain trauma studies.

In the present disclosure, the microfluidic cell culture device is used for cell cultivation. The term “cell cultivation” as used herein refers to a process of growing cells, having a growth rate, from a small number to a larger number, in an artificially created environment under controlled conditions. Said controlled conditions are suitable for an optimal growth of cells and include a suitable container for growth, growth media (comprising nutrient feed, growth factors, hormone), and physicochemical parameters (such as pH, osmotic pressure, humidity, temperature, sterile conditions). Moreover, the small number of cells (namely, inoculum) are provided as input for the device and the larger number of cells (namely, cultured cells) are received as output. It will be appreciated that new cells may be added after a desired growth of cells is achieved. Optionally, cells may be cultivated in the growth media and one or more analytes. Optionally, the one or more analytes are added to the cell culture to monitor the effects of the one or more analytes on the cells and/or various cellular processes. Optionally, the one or more analytes may include, but do not limit to, therapeutics, drugs, pollutants and toxins. Optionally, the various cellular processes include, but do not limit to, a growth and regeneration, a differentiation, a motility, a division, an adhesion, a secretion, a death, a genotype, a phenotype, a metabolism.

Throughout the present disclosure, the term “microfluidic cell culture device” as used herein refers to a device that mimics natural biological environment and creates an in vitro micro-physiological environment for biological material, such as a cell, therein. In this regard, the microfluidic cell culture device incorporates microfluidic flow, vasculature and barriers to provide living cell-like conditions for the growth of the biological material. Notably, the biological material have an innate mechanism to grow by multiplying cells in optimum conditions of growth. The growth media and the microfluidic cell culture device provide such optimum conditions, for example, suitable temperature, pH, nutrients, moisture, gaseous exchange, and so forth, for the growth of the biological material. Moreover, the microfluidic cell culture device comprises all the components necessary for cultivating the biological material in a single, compact, readily handled unit. The microfluidic cell culture device could be employed for growing cells and studying cellular interactions, cellular wounding and healing mechanisms, and so forth. Additionally, such microfluidic cell culture devices may be used to analyse effects of various pharmacological or toxicological compounds on the cells.

Moreover, the microfluidic cell culture device of the present disclosure may be of various shapes (such as round or planar), sizes and fabrication to suit a variety of cell cultures and cellular processes. Optionally, the microfluidic cell culture device may be of different shapes, such as a square prism, a rectangular prism, a cylinder, a sphere, a disc, a slide, a chip, a film, a plate, a pad, a tube, a strand, a box, and the like. Optionally, the microfluidic cell culture device may be of different sizes to allow easy manipulation of the content therein, and be compatible with a variety of standard lab equipment such as microtiter plates, multichannel pipettors, microscopes, inkjet-type array spotters, photolithographic array synthesis equipment, array scanners or readers, fluorescence detectors, infra-red (IR) detectors, mass spectrometers, thermocyclers, high throughput machinery, robotics, and the like.

Optionally, the microfluidic cell culture device is manufactured using a fabrication material selected from any of: polydimethylsiloxane (PDMS), Flexdym™ polymer, thiol-ene polymer, UV-curable epoxy resin-based photoresist, PMMA, polystyrene, PLGA, soft thermoplastic elastomer (sTPE), styrenic block copolymer (BCP), SU-8 polymer, or any combination thereof. Notably, the microfluidic cell culture device is manufactured from a fabrication material that is typically suitable for cell culture, waterproof, and strong enough to withstand effects of various biological and/or biochemical processes during use. Optionally, said fabrication material provides an optimum flow rate, microfluidics, physiological conditions and so forth thereby mimicking the natural function of a biological material. In an example, the fabrication material of the microfluidic cell culture device allows consistent exchange of gas molecules or small molecules between two liquids or a liquid and a gas therethrough without direct contact. Such indirect diffusion of molecules keeps the cells fresh and alive for a longer period. The microfluidic cell culture device is manufactured using multiple layers of fabrication material. Typically, the multiple layers of said fabrication material has an adequate thickness to hold a weight of the growing cells and carry out various processes.

Optionally, manufacturing and processing techniques may include, but not limit to, injection moulding, over moulding, three-dimensional printing, photolithography, and so on. In an example, polydimethylsiloxane (PDMS), a mineral-organic polymer containing carbon and silicon, is used as the fabrication material for the manufacture of the microfluidic cell culture device. Notably, PDMS is known for its biocompatibility, transparency, flexibility, gas permeability, low solubility and low surface tension. In this example, manufacturing the microfluidic cell culture device includes mixing the PDMS base monomer with a cross-linking agent (for curing of PDMS), pouring the resulting mixture into a micro-structured mould of desired shape and size, and subjecting thereof to an optimum temperature to obtain an elastomeric replica of the mould. Moreover, multiple layers of PDMS are stacked on top of each other to result in a structure with complex geometry enabling addition of membranes, barriers, conduits, and other such potential things that may be integrated as desired. The multiple layers are bonded together using a plasma bonding or an adhesive material. Moreover, optionally, the microfluidic cell culture device is plasma-treated, flushed with ethanol and a buffer to remove air bubbles, and applied a coating solution containing a cell adhesive material such as a collagen, a fibronectin, a laminin, a hyaluronic acid, other matrix proteins, or a combination thereof. Normally, the microfluidic cell culture device is incubated for a predefined time after which the coating solution is removed, and the microfluidic cell culture device is washed before adding the biological material. In an example, the microfluidic cell culture device may comprise 6 layers of PDMS, each with a thickness in a range between 100-3000 micrometre (μm). The upper limit could be 1 or 2 cm in an example. The thickness of each layer of the microfluidic cell culture device can be for example from 100, 500, 1000, 1500, 2000 or 2500 μm up to 500, 1000, 1500, 2000, 2500 or 3000 μm. In an alternative example, other poly silicate material or plastics may be used to manufacture the microfluidic cell culture device.

It will be appreciated that the aforementioned microfluidic cell culture device is a single unit, and a plurality of such units can be combined to form a high throughput well plate format chips or arrays of units as per the ANSI well plate format or other such high throughput system. Beneficially, such high throughput systems allow performing (and analysing) tissue wound and healing models in multiples. Moreover, such high throughput systems allow efficient automation as well as robotic handling.

Optionally, the microfluidic cell culture device may be designed as a clear plate or chip for improved optical clarity or as coated plate or chip for use in fluorescence and/or luminescence studies. Beneficially, the microfluidic cell culture device may be used to fit with a variety of standard multi-well cell culture plates for various applications ranging from cloning, incubations, screening, and so forth. Typically, the standard mufti-well cell culture plates have defined outer dimensions, generally in a 2:3 rectangular matrix. The standard mufti-well cell culture plate may be selected from, for example, the standard 6, 12, 24, 48, 96, 384, 1536 and 3456-well cell culture plate comprising a 2×3, 3×4, 4×6, 6×8, 8×12, 16×24, 32×48 and 48×72 matrix of wells for high throughput analysis. As a result, the well dimensions, diameter and distances between the wells of the multi-well cell culture plates is also defined as per the industry standards. Optionally, the microfluidic cell culture device of the present disclosure can be produced in large quantities in sheets and punched into a desired shape, such as spheres, to fit the wells of the mufti-well cell culture plates. It will be appreciated that the dimensions and volumes enclosed thereby cannot be larger than the well itself. Moreover, the microfluidic cell culture device should have same outer dimensions as per its use with the different multi-well cell culture plates formats.

Furthermore, the dimensions of the microfluidic cell culture device in the specific well plate format may be defined based on a maximum length of the mufti-well cell culture plate format and a maximum width of the mufti-well cell culture plate format. The maximum length is calculated as a multiple of the inter-well distance (i.e. three wells in the microfluidic cell culture device device) minus some minimum thickness of the microfluidic chamber wall for the sake of material integrity as it is relevant from a manufacturing and usability standpoint. Similarly, the maximum width is calculated as a multiple of the inter-well distance (i.e. 1 well in the microfluidic cell culture device device) minus some minimum thickness of the microfluidic chamber wall.

In an example, the microfluidic cell culture device may be used with a standard 384 well plate format (ANSI SLAS 4-2004 (R2012) Microplate standard for well positions). In this regard, the internal dimensions of the microfluidic cell culture device may for example be 3.7 mm×12.7 mm (width×length). Moreover, the internal dimensions of the microfluidic cell culture device would decrease roughly by a factor of two and three for the 1536 and 3456 well plate formats, respectively. In this regard, for use in a 3456 well plate format device, the internal dimensions of the microfluidic cell culture device could be 1.0 mm×4.0 mm. Furthermore, the height of the microfluidic cell culture device may range from 5 mm to 20 mm. It will be appreciated that the length of the microfluidic cell culture device may be increased if additional wells were to be added in the multi-well cell culture plate format having outer dimensions as per the ANSI standard. It will be appreciated the external dimensions of the microfluidic cell culture device will also follow the standard 384 well plate format (Well Plate Footprint Dimensions—ANSI/SLAS 1-2004 (R2012)).

Optionally, the cell cultivation is a three-dimensional cell cultivation. The term “three-dimensional cell cultivation” as used herein refers to cultivation of single or multiple cells under controlled conditions to grow and interact with the surroundings thereof in three dimensions similar to the in-vivo cell growth. The three-dimensional cell cultivation uses an acellular three-dimensional scaffold (or substrate) or a scaffold-free liquid suspension media for growing cells. The three-dimensional substrate includes, but is not limited to, hydrogel matrix, porous membranes and solid scaffolds. Beneficially, the three-dimensional cell cultivation provides three-dimensional cell cultures with more contact space for cell adhesion and intracellular signalling. Beneficially, the three-dimensional cell cultures may be used in drug discovery, tissue engineering and pharmacological and toxicological research, for example, analysing effect of various substances on the cells.

Optionally, the three-dimensional cell cultivation is a nervous system-mimicking cell cultivation. In an implementation, the microfluidic cell culture device could be employed to model the human nervous system. In this regard, said device could be designed to culture brain cells, and/or induce a wound (or damage) to the cells and subsequently allow other cells to interact and overcome the damage caused.

Optionally, the biological material is selected from at least one of: a cell, a cell spheroid, an organoid, or a tissue. The biological material is selected from a group of cells having a growth rate. The biological sample is typically isolated from its natural environment under optimum conditions. Optionally, the methods of isolation of the biological sample is selected from conventional methods of cell isolation, known in the art, and thus has not been described in detail herein for the brevity of the present disclosure. Optionally, the biological material is a cell line, a monolayer cultured cell, cells embedded in a matrix, an organ, a microorganism culture or a combination thereof. Optionally, the biological material is an organoid or a spheroid. Optionally, size of the biological material may range between 1 and 6 mm. Typically, the size of the biological material may be from 1, 1.5, 2, 2.5, 3, 3.5, 4, 4.5, 5 or 5.5 mm up to 1.5, 2, 2.5, 3, 3.5, 4, 4.5, 5, 5.5 or 6 mm. It will be appreciated that the biological sample may be a single cell or multiple cells of same or different types co-cultivated.

Optionally, the cells are selected from at least one of: nerve cells, glia cells, vascular endothelial cells, fibroblasts, muscle cell, astrocytes, pericytes, or any combination thereof. The nerve cells are the main components of the nervous tissue in majority of organisms. The nerve cells or neurons are electrically excitable cells which communicate with other cells via special connections called synapses. Nerve cells play an important role in the nervous system, such as responding to stimuli for example touch, sound, light, smell, or taste affecting the sensory organs, and send signals to spinal cord or brain; receiving signals from the spinal cord and brain to control muscle contractions or glandular output; and connecting nerve cells to other nerve cells in vicinity. Glia cells are the non-neuronal cell that maintain homeostasis, form myelin, and provide support, protection and oxygen to nerve cells, neurotransmission, and synaptic connections. Astrocytes are a type of glia cells in the brain and spinal cord, that provide biochemical support to endothelial cells that form blood-brain barrier, nutrients to the nervous tissue, maintenance of extracellular ion balance, regulation of cerebral blood flow, and repair and scarring process of the brain or spinal cord following infection or injury. The pericytes wrap around the endothelial cells and maintain homeostatic and hemostatic functions in the brain and sustain the blood-brain barrier. The fibroblast plays a critical role in wound healing. It will be appreciated that cells, same or different types, interact with each other to effect proper functioning of various physiological processes. Optionally, the cells may be derived from microbes, viruses, fungi, plants, or animals to study various cellular processes thereof.

The microfluidic cell culture device for cell cultivation comprises two or more media reservoirs. The term “media reservoir” as used herein refers to a storage compartment, vessel, container and so forth for storing the growth media for cell cultivation. Each of the two or more media reservoirs comprises a top end and a bottom end opposite the top end enclosing a predefined volume therebetween for containing a liquid media, such as the growth media. Moreover, each of the two or more media reservoirs have a first opening on the top end and a second opening on the bottom end, wherein the first opening allows adding fresh liquid media into the two or more media reservoirs, and the second opening allows dispensing the liquid media for cell culture. It will be appreciated that the liquid media provides an artificial growth environment which mimics the natural environment for the growth of the cells. Typically, the liquid media comprises necessary nutrients, growth factors, and hormones for cell growth, as well as regulates the pH and the osmotic pressure of the cell culture. Optionally, the liquid media may contain a mixture of amino acids, glucose, salts, vitamins, and other nutrients, and is available as a powder or in a liquid form.

Moreover, the microfluidic cell culture device comprises the first microfluidic chamber having the non-planar surface and the second microfluidic chamber being the pressure chamber. The first microfluidic chamber and the second microfluidic chamber resemble two continuous, operatively coupled parts (such that a bottom end of one line a top end of the other) of a microfluidic chamber for culturing various types of biological material, i.e. cells or cells-containing bodies. Each of the first microfluidic chamber and the second microfluidic chamber comprises a top end and a bottom end opposite the top end enclosing a respective predefined volumes therebetween. Moreover, the second microfluidic chamber atop the first microfluidic chamber such that the bottom end of the second microfluidic chamber leads to (or lines with) the top end of the first microfluidic chamber, and the top end of the second microfluidic chamber faces an open top surface of the microfluidic cell culture device. It will be appreciated that the top end of the second microfluidic chamber is not open but closed, and has a pressure inlet coupled to a pressure actuator. Moreover, the second microfluidic chamber is typically devoid of any matter and thus functions as a vacuum chamber. It will be appreciated that the microfluidic chamber is arranged between two or more media reservoirs, wherein the first microfluidic chamber is arranged corresponding (namely, in line with) to the second opening on the bottom end of each of the two or more media reservoirs and the second microfluidic chamber is arranged corresponding (namely, in line with) to the first opening on the top end of each of the two or more media reservoirs.

Optionally, the second microfluidic chamber is filled with a gas or a liquid. In this regard, the second microfluidic chamber is used in conjunction with the pressure actuator. The pressure actuator is used to actuate fluid flow with pressure. Optionally the pressure may be any of: an air pressure, liquid pressure, mechanical pressure, a combination thereof, or any other type of pressure, to study the effects of fluid pressure on the cell culture. Moreover, fluid pressure may be used to study any type of cell cultures where the cells are being subjected to mechanical or fluid pressure (i.e., air or liquid pressure). Moreover, standard pressure could be achieved with precise pressure control either in pneumatic or liquid actuated systems. Optionally, the second microfluidic chamber is used in conjunction with a pneumatic pressure actuator that employs air for actuation. Optionally, the second microfluidic chamber is filled with a gas or a liquid using the pressure actuator to create a pneumatic or hydraulic pressure, respectively. In high throughput format, the pressure actuator could be combined to form a larger unit comprising multiple pressure channels, as is desired for it to be able to actuate multiple microfluidic cell culture devices at once with a single pressure actuator. Optionally, the pressure actuator could be as low tech as a syringe or a fully automated pressure actuator.

It will be appreciated that the flexible non-porous membrane is air permeable, and the pneumatic pressure may be lost over time, therefore, at this stage, one or more analytes could be added to the cell culture that is damaged or stressed with said pneumatic pressure for studying cellular processes therewith. Moreover, since the pneumatic pressure may be lost over time, hydraulic pressure may be selected for actuation of the flexible non-porous membrane for experiments running for a longer duration of time. Alternatively, the fluid actuation could be performed by flexible mechanical levers. In this regard, the flexible mechanical levers may be cast into the microfluidic cell culture device which would be able to provide the same pressure in each of the microfluidic cell culture device if the same mechanical action is performed by each of the microfluidic cell culture device.

The term “non-planar surface” as used herein refers to a surface having a three-dimensional quality. Moreover, the bottom end of the first microfluidic chamber comprises the non-planar surface. The non-planar surface is typically like the rough surface with non-planar features. The non-planar surface typically provides space for positioning and guidance for growing the biological material. Specifically, the non-planar surface enables mimicking nervous system cell cultures, and/or help position organoids in a desired location where the application of pressure is best i.e. at the centre of the first microfluidic chamber, to study nervous system model.

Optionally, the non-planar surface has one or more pits. The term “pits” as used herein refers to miniaturised wells for enabling cell cultivation therein. The one or more pits have a volume for holding a biological sample and a predefined amount of culture media for growth thereof. Optionally, the one or more pits are embedded in the first microfluidic chamber. Optionally, two adjacent pits are separated by a thickness of walls of each of the pits. Optionally, the one or more pits are of a predefined cross-section. Typically, the predefined cross-section includes a shape, a size, arrangement, and so forth. Optionally, the one or more pits are shaped as contours, pits, grooves, ridges, posts, spikes, striations, or half-spheres. Optionally, the one or more pits have one of: a conical cross-section, a cuboidal cross-section, a cubical cross-section, a cylindrical cross-section, a spherical cross-section, an elliptical cross-section, a hexagonal cross-section, a polygonal cross-section, and so forth. Optionally, the one or more pit has a flat bottom, a bottom with minimal rounded edges (concave), a V-shaped bottom or a U-shaped bottom.

Optionally, the one or more pits has a height in a range between 0.3-3.0 mm and a thickness in a range between 0.1-3.0 mm. The height of the one or more pits may typically be from 0.3, 0.6, 0.9, 1.2, 1.5, 1.8, 2.1, 2.4 or 2.7 millimetre (mm) up to 0.6, 0.9, 1.2, 1.5, 1.8, 2.1, 2.4, 2.7 or 3.0 mm, and the thickness of the one or more pits may typically be from 0.3, 0.6, 0.9, 1.2, 1.5, 1.8, 2.1, 2.4 or 2.7 mm up to 0.3, 0.6, 0.9, 1.2, 1.5, 1.8, 2.1, 2.4, 2.7 or 3.0 mm. It will be appreciated that said multitude of shapes and sizes should allow for expansion and accommodation of the cells in the one or more pits during growth, while still maintaining the flow of the media (and/or one or more analytes) therethrough to keep the cells alive.

It will be appreciated that in order to achieve cell cultivation in a high throughput manner, the number of pits could be as high as possible. In such case, the thickness of each of the pits may be reduced to increase the number of pits per square area of the first microfluidic chamber. However, increasing the number of pits with reduced thickness may result in lower quality microfluidic cell culture device and ineffective cell cultivation. In an example, the higher number of pits may risk material integrity of the microfluidic cell culture device at the time of moulding and demoulding, and inaccurate placement of microfluidic cell culture device in standard mufti-well cell culture plates, such as for example a misoriented placement leading in misloading of the biological material to a corner of the bottom of the pit and not the centre of the pit. In this regard, the microfluidic cell culture device of the present disclosure provides for example 4 to 25 pits in the first microfluidic chamber to generate a high-throughput cell cultivation without risking the material integrity of the microfluidic cell culture device.

It will be appreciated when the number of pits is more than one, the pits are arranged in rows which can be arranged in an array unit. Optionally, the array unit may be selected from a square array unit, a rectangular array unit, round array unit, or a hexagonal array unit. More optionally, the square array unit comprises four or more pits in a matrix of pits, while a hexagonal array comprises six or more pits in a matrix of pits. Optionally, the square array unit may have one of: a 2×2 matrix comprising 4 pits, a 3×3 matrix comprising 9 pits, a 4×4 matrix comprising 16 pits, a 5×5 matrix comprising 25 pits, a 10×10 matrix comprising 100 pits, and so forth. Optionally, the rectangular array unit or the round array unit may comprise one or more pits in a matrix of pits. In an example, the rectangular array unit comprises a 1×8 matrix comprising 8 pits. It will be appreciated that when the one or more pits are shaped as grooves or ridges, the groove or ridge-shapes pits could be arranged in lines each parallel to one another or at an angle with each other. In an example, 8 groove or ridge-shapes pits could be arranged as 1×8 matrix of pits.

Furthermore, the microfluidic cell culture device comprises the flexible non-porous membrane that separates the first microfluidic chamber and the second microfluidic chamber, wherein the flexible non-porous membrane is arranged opposite to the non-planar surface of the first microfluidic chamber. The term “membrane” as used herein refers to a planar sheet having a first face and a second face. The flexible non-porous membrane is arranged between the first microfluidic chamber and the second microfluidic chamber. In this regard, the flexible non-porous membrane is arranged between the first microfluidic chamber and the second microfluidic chamber such that the first face of the flexible non-porous membrane is facing the non-planar surface of the first microfluidic chamber and the second face of the flexible non-porous membrane is facing the open top surface of the microfluidic cell culture device. In other words, the first microfluidic chamber, the flexible non-porous membrane and the second microfluidic chamber are arranged as a stack. Beneficially, the flexible non-porous membrane enables fluid actuation, when a pressure is applied thereto.

Optionally, the flexible non-porous membrane has a plunger area. The term “plunger area” as used herein refers to a protrusion appearing from the flexible non-porous membrane. It will be appreciated that the plunger area is designed to press against a corresponding surface and apply pressure thereto. Optionally, the height of the plunger area is 50-500 micrometres. The height of the plunger area may typically be from 50, 100, 150, 200, 300 or 400 micrometre (μm) up to 100, 150, 200, 300, 400 or 500 μm.

Optionally, the plunger area comprises one or more features arranged to face the non-planar surface. Specifically, the flexible non-porous membrane is arranged between the first microfluidic chamber and the second microfluidic chamber such that the plunger area faces the non-planar surface of the first microfluidic chamber. More specifically, the one or more features protrude from a side of the plunger area that faces the non-planar surface of the first microfluidic chamber. The one or more features are designed to apply the pressure at specific sites on the opposing surface thereto, such as the one or more pits on the non-planar surface of the first microfluidic chamber. Alternatively, the one or more features may function as a probe or needle which can be placed inside a tumour organoid growing inside one or more pits of the first microfluidic chamber, allowing for sample collection from inside the growing tumour.

Optionally, the one or more features correspond to the one or more pits. The one or more features and the one or more pits are arranged opposite to each other facing each other. Furthermore, a feature is aligned with a pit to enable exerting pressure at specific sites, i.e. the corresponding pit. Therefore, a feature is either a negative of a corresponding pit or designed in a manner so as to slide in the corresponding pit. Optionally, the one or more features are perpendicular to the correspondingly arranged one or more pits, and are able to slide in the corresponding one or more pit. Optionally, the one or more features are complimentary to the correspondingly arranged one or more pits, and are negatives of the corresponding one or more pit. Optionally, the application of pressure via the one or more features of the plunger area is limited to not cover the whole area of the one or more pits on the non-planar surface of the first microfluidic chamber under the plunger area or on e or more features by grooves or features, thus allowing cells growing in the one or more pits to experience less pressure.

In this regard, therefore, optionally, the one or more features are of a predefined cross-section significantly complimentary to the predefined cross-section of the one or more pits. Typically, the predefined cross-section of the one or more features includes a shape, a size, arrangement, and so forth. Optionally, the one or more features are shaped as contours, pits, grooves, ridges, posts, spikes, striations, or half-spheres. Optionally, the one or more features have one of: a conical cross-section, a cuboidal cross-section, a cubical cross-section, a cylindrical cross-section, a spherical cross-section, an elliptical cross-section, a hexagonal cross-section, a polygonal cross-section, and so forth. Optionally, the one or more feature has a flat bottom, a bottom with minimal rounded edges (convex), a V-shaped bottom or a U-shaped bottom. Optionally, the one or more features has a height in a range between 0.3-3.0 mm and a thickness in a range between 0.1-3.0 mm. The height of the one or more pits may typically be from 0.3, 0.6, 0.9, 1.2, 1.5, 1.8, 2.1, 2.4 or 2.7 millimetre (mm) up to 0.6, 0.9, 1.2, 1.5, 1.8, 2.1, 2.4, 2.7 or 3.0 mm, and the thickness of the one or more pits may typically be from 0.1, 0.3, 0.6, 0.9, 1.2, 1.5, 1.8, 2.1, 2.4 or 2.7 mm up to 0.3, 0.6, 0.9, 1.2, 1.5, 1.8, 2.1, 2.4, 2.7 or 3.0 mm. In an example, when the one or more pits are cuboidal in cross-section, the corresponding one or more features are cuboidal in cross-section, such that the cross-section of the one or more features are lower that than the cross-section of the one or more pits to enable passing of a feature into the corresponding pit. In another example, the one or more pits with concave shape have corresponding one or more features with convex shape for sliding therein. Preferably, one or more features with a flat bottom or a bottom with minimal rounded edges (concave) are easier to manufacture compared to other types. Also, for pressing on a striated non-planar surface, one or more features with a flat bottom (rectangular) is the preferred geometry to get even pressure across all pits. Moreover, one or more features with a short height and a concave end are preferred, though one or more features with a hexagonal or octagonal cross-section and corresponding one or more pits may be preferred if the cell culture is of an organoid as the slight edges of the one or more features would retain the roundish organoid in place in the one or more pits. Optionally, one or more features with a V-shape bottom (or conical end or a spike) could serve as a needle for injecting one or more analytes, such as drugs or other materials, into an organoid. Alternatively, said feature could be used as a syringe for investigating the chemical status inside an organoid. Moreover, in an embodiment, said feature could be combines with one or more electrodes to result in different simulation models.

Optionally, when number of features is more than one and the features are arranged in form of an array unit selected from one of: a square array unit, a rectangular array unit, a round array unit, or a hexagonal array unit. In this regard, it will be appreciated that in order for the one or more features to correspond to the one or more pits, the number of features should be the same as the number of pits. Moreover, the arrangement of the one or more features should be same as the arrangement of the one or more pits. More specifically, if the features are arranged for example in square array unit, rectangular array unit, round array unit, or hexagonal array unit, then the pits should also be arranged in square array unit, a rectangular array unit, a round array unit, or a hexagonal array unit. Optionally, the square array unit comprises four or more features in a matrix of features, while a hexagonal array comprises six or more features in a matrix of features. Optionally, the square array unit may have one of: a 2×2 matrix comprising 4 features, a 3×3 matrix comprising 9 features, a 4×4 matrix comprising 16 features, a 5×5 matrix comprising 25 features, a 10×10 matrix comprising 100 features, and so forth. Optionally, the rectangular array unit or the round array unit may comprise one or more features in a matrix of features. In an example, the rectangular array unit comprises a 1×8 matrix comprising 8 features. It will be appreciated that when the one or more features are shaped as grooves or ridges, the groove or ridge-shapes features could be arranged in lines each parallel to one another or at an angle with each other. In an example, 8 groove or ridge-shapes features could be arranged as 1×8 matrix of features.

Optionally, the flexible non-porous membrane with features could be made by casting polydimethylsiloxane (PDMS), Flexdym or other polysilicone material into a thin film with the mould having depressions of desired shape and position. Said thin film in the mould when incubated and retracted from the mould would then become the protruding features on the flexible non-porous membrane, that are configured to apply the pressure on the one or more pits and the contents therein. Optionally, the flexible non-porous membrane is composed of a material that is biocompatible. Optionally, the one or more features applies a lateral pressure to the cells when the vertical pressure from the flexible non-porous membrane is applied. Moreover, the fabrication material of the features may be selected so that a desired pressure is reached when the flexible non-porous membrane applies a certain pressure.

Optionally, the biological material (namely, cells or cell-containing bodies) may be added into the one or more pits of the first microfluidic chamber in suspension, one at a time or in a mixture. Optionally, the biological material may be loaded into the one or more pits of the first microfluidic chamber while being suspended in the liquid media or buffer solutions containing the biological material. The biological material in the liquid are incubated for a predefined incubation period of time (based on the type of cells used) to produce a three-dimensional cell culture in the one or more pits of the first microfluidic chamber. Optionally, the predefined incubation period may be for example, of 4 days, in the one or more pits. Said period of growth results in generation of the three-dimensional cell culture in the one or more pits. When the cell culture is ready, the second microfluidic chamber is either added with air or liquid to apply pressure to the flexible non-porous membrane for actuation thereof. The actuation of the flexible non-porous membrane can be kept for seconds, minutes or hours based on the type of cells or as the test may require. The actuation of the flexible non-porous membrane results in the one or more features of the flexible non-porous membrane to damage or stress the cell culture in the one or more pits of the non-planar surface of the first microfluidic chamber. Optionally, the cells or cell-containing bodies can be loaded at a centre of the one or more pits of the non-planar surface of the microfluidic cell culture device. The cells or cell-containing bodies can subsequently be pressed with the flexible non-porous membrane having one or more features with a concave indentation that allows the cells or cell-containing bodies to stay in place while being pressed which also distributes the force more evenly.

Beneficially, the flexible non-porous membrane actuation is suitable to model the human nervous system. The nervous system model may have ridged or striated non-planar surface of the first microfluidic chamber to provide direction for the nerve cells to grow. Furthermore, in order to materialize said device which mimics the natural function of an organ or tissue, eligible fabrication materials known for their microfluidics and flow dynamic properties and suitable for cell culture are employed.

Optionally, the fabrication material of the non-planar surface is selected based on a structural integrity thereof, when the second microfluidic chamber is at a predefined pressure. Specifically, the fabrication material of the non-planar surface must lose structural integrity thereof when the second microfluidic chamber reaches a predefined pressure. More optionally, the fabrication material of the non-planar surface could crumble, smash, tear, and so forth, under the pressure exerted by the second microfluidic chamber. Specifically, the fabrication material of the non-planar surface must break or structurally deform at a certain load or predefined pressure from the second microfluidic chamber. In this regard, when simulating the nerve cell damage, an aim might be to apply enough pressure to the nerve cells to cause severe cell damage. In this regard, the plunger area comprising the one or more features may be required to reach the bottom of the one or more pits of the non-planar surface. Moreover, the one or more features correspond to the one or more pits, and function as a negative therefor. Alternatively, the one or more features and the one or more pits could be fabricated using a soft material that breaks when a sufficient pressure is applied to. Moreover, such soft material acts as a growth guide for the nerve cells but deforms when applied pressure thereto, thereby, allowing enough pressure to be exerted on the nerve cells. Furthermore, using a soft material enables different form factors of the one or more features and the one or more pits to be locked into each other to cause damage to the nerve cells once the soft material is crushed or broken.

Optionally, the fabrication material of the non-planar surface is selected based on a desired predefined pressure to be exerted on a biological material, when the second microfluidic chamber is at a predefined pressure. It will be appreciated that the fabrication material may be selected based on the type of biological material being cultured. Notably, different biological materials have different structural integrity and require different levels of pressure for causing damage or stress thereto. Moreover, the fabrication material of the non-planar surface must also enable cell growth thereon.

Optionally, the non-planar surface is fabricated from at least one of: a silicone material, a hydrogel material. Moreover, the non-planar surface is fabricated from a soft and thin material. The hydrogel material may be natural, synthetic or hybrid materials, including, but not limited to, collagen, fibrin, alginate, polyacrylamide, polyethylene glycol, hyaluronic acid and polypeptides. Optionally, the hydrogels may function as a bio-ink to print cells either in a layer-by-layer manner from a 2D substrate or directly in three-dimensions within another hydrogel. Beneficially, the silicone material or the hydrogel material may used as cell culturing substrates that allow expansion of cells and offer an atmosphere for regular cell growth in-vitro. Additionally, beneficially, said materials provide a hydrophobic barrier and a high oxygen permeability to the growing cells. Moreover, said materials are suitable for electrical actuation. Furthermore, it will be appreciated that due to the softness of said materials, the cell cultures grow in three-dimensions, thereby better mimicking the biological milieu, and not with a flattened shape, abnormal polarization, and loss of differentiated phenotype as with conventional plastic material or glass material.

Optionally, the first microfluidic chamber has a non-planar surface arranged on top of a planar surface, and wherein the planar surface is fabricated from at least one of: a glass material, a plastic material. In this regard, the glass material and the plastic material provide a flat, unphysiologically stiff surfaces onto which non-planar surfaces are arranged for allowing a three-dimensional growth of the cells thereon. Moreover, the glass material and the plastic material provide a required firmness for cell disruption when pressure is applied by the Flexible non-porous membrane.

Optionally, the first microfluidic chamber has an adhesive coating, the adhesive coating being configured to adhere the biological material on to the adhesive coating and cultivate the biological material with perfusion. The term “adhesive coating” as used herein refers to a coating that enhances cell attachment on a surface, such as the first microfluidic chamber, and preferably, the non-planar surface of the first microfluidic chamber. Optionally, the adhesive coating lines the first microfluidic chamber on the non-planar surface thereof. More optionally, the adhesive coating lines the silicone material, or a hydrogel material used for fabrication of the non-planar surface. Beneficially, the adhesive coatings usually enhance or promote attachment of cells thereto, and allowing the cells to adhere to the surface, move on it, proliferate, and the like.

Optionally, the non-planar surface has electrodes or sensors imbedded therein. Besides the actuation of the flexible non-porous membrane and one or more features thereof to damage or stress the cell culture in the one or more pits, the non-planar surface could further be stimulated with electrodes placed on the opposite ends of the first microfluidic chamber. The electric current from the electrodes enhances the cell growth and promote the healing and regeneration of the cells. Beneficially, electrodes may help speed wound healing by increasing capillary density and perfusion, improving wound oxygenation, and encouraging granulation and fibroblast activity. It will be appreciated that the additional stimulus on the opposite ends of the non-planar structure enables effective mimicking the basis of our nervous system cell culture.

Furthermore, the microfluidic cell culture device comprises the one or more microfluidic channels connecting the first microfluidic chamber to the two or more media reservoirs. The term “microfluidic channel” as used herein refers to a capillary arrangement for supplying a fluid (such as the liquid media) from the two or more media reservoirs to the microfluidic cell culture device. The microfluidic channel supplies the media (and/or one or more analytes) to the first microfluidic chamber, when in operation. Moreover, the microfluidic channel is configured to supply different concentrations of the one or more analytes to each of the pits of the first microfluidic chamber.

Optionally, the microfluidic channel is integrated into the microfluidic cell culture device, i.e. in between the PDMS layers. Optionally, the microfluidic channels may be manufactured using the same fabrication material as that of the layers of the microfluidic cell culture device or using a different material, for example Teflon, glass, fibre optic, and so forth. More optionally, the multiple layers of the fabrication material of the microfluidic cell culture device, with microfluidic channels integrated therein, are tightly sealed (or adhered to each other) to prevent fluid flowing through the microfluidic channel from flowing out thereof.

Optionally, the one or more microfluidic channels pass through the flexible non-porous membrane. Optionally, the one or more microfluidic channels pass through the plunger area of the flexible non-porous membrane. The one or more microfluidic channels in the flexible non-porous membrane are configured to deliver a predefined amount of liquid media to the cell culture in the first microfluidic chamber. Moreover, the one or more microfluidic channels in the flexible non-porous membrane may be configured to apply to the cell culture at least one of: molecules, proteins, antibodies, lipids, lipid particles, micelles, cell fragments, or whole cells or cell-containing structures. Optionally, applying said compounds could be done for the purpose of staining and imaging, drug testing, and/or to provide stimulus to cells. Moreover, the one or more microfluidic channels in the flexible non-porous membrane could be used to apply fluorescent antibodies instead of cell lines expressing fluorescent proteins for a real-time or time-lapse imaging. In this regard, optionally, the one or more microfluidic channels in the flexible non-porous membrane may be used to create an array for staining one or more pits with multiple fluorescent markers at the same time while the cell culture is alive. This allows one to study the cell culture system in real-time, collecting multiple time points from a single experiment, and staining against multiple molecules of interest. Thereby, decreasing the number of experiments needed for an extensive dataset.

Optionally, the one or more microfluidic channels in the flexible membrane have one or more outlets. Optionally, the one or more outlets pass through the plunger area of the flexible non-porous membrane. The one or more outlets typically protrude from the main one or more microfluidic channels to open into different parts of the microfluidic cell culture devices or outside thereof. It will be appreciated that the one or more microfluidic channels runs vertically between the one or more outlets and the first microfluidic chamber and through the flexible non-porous membrane, and the main one or more microfluidic channels itself runs horizontally between the two or more reservoirs and the first microfluidic chamber.

More optionally, each of the one or more outlets has a first end and a second end, and wherein the first end opens in to the first microfluidic chamber, and the second end opens in to at least one of: a side corresponding to the top end of the second microfluidic chamber; one or more biological material reservoirs; and an external outlet, such that the one or more microfluidic channels connects the first microfluidic chamber to the one or more outlets. Moreover, the first microfluidic chamber is connected to the one or more outlets, through the one or more microfluidic channels, running inside the flexible non-porous membrane and up vertically parallel to the second microfluidic chamber. It will be appreciated that the first end of each of the one or more outlets opens into the first microfluidic chamber to provide the liquid media from the two or more media reservoirs to the first microfluidic chamber. Optionally, the second end of the one or more outlets that opens outside of the microfluidic cell culture device are configured for obtaining samples from the damaged biological material (cells or tissues) after exerting pressure. Optionally, the second end of the one or more outlets that opens outside of the microfluidic cell culture device are configured for adding analytes or similar or different types of biological material (cells or tissues) to the damaged biological material (cells or tissues). It will be appreciated that the second end of the one or more outlets opens outside the second microfluidic chamber to avoid gas or fluid from flowing between the first microfluidic chamber and the second microfluidic chamber when pressure is applied to the second microfluidic chamber with the purpose of actuating the flexible non-porous membrane. Alternatively, the motion in the one or more microfluidic channels and the liquid media could be achieved manually by tilting the microfluidic cell culture devices. Furthermore, the second end of the one or more outlets enable harvesting the cultured cells, removing the used liquid media, and clearing dead cells and debris from the cell culture. Moreover, additional cells could be added to the cell culture once the tissue is damaged or stressed with pressure. Said additional cells include, but do not limit to, immune cells such as monocytes, macrophages, lymphocytes or any other type of tissue-appropriate immune cell. Furthermore, the one or more outlets of the one or more microfluidic channels in the flexible non-porous membrane may be configured to acquire effluent samples from the damaged cells, even while they are under pressure, to be analysed. Optionally, the one or more outlets are used to introduce chemical or biological agents onto the cell culture or into the tissue.

Optionally, the one or more features as well as the one or more pits may cause an image blur at certain areas of the image as the difference in material density compared to the liquid media causes light to bend irregularly across the image field.

Optionally, the grown, wounded or healed cells may be harvested from the external outlet. Alternatively, harvesting of cells may be achieved by introducing a needle or syringe within the first microfluidic chamber to withdraw the cells.

Optionally, the two or more media reservoirs or one or more biological material reservoirs is connected to a pressure actuator. Optionally, the pressure actuator connected with the two or more media reservoirs or one or more biological material reservoirs could be same as coupled to the second microfluidic chamber or different therefrom. The pressure actuators enable motion in the liquid media and the buffer solution containing biological material to be circulated in the two or more media reservoirs or one or more biological material reservoirs, respectively. Moreover, pressure actuation in the two or more media reservoirs or one or more biological material reservoirs also results in feeding the liquid media and the buffer solution containing biological material into the one or more pits of the first microfluidic chamber for the cell culture. In an example, the pressure actuator may result in dispensing 10 μl of the liquid media in every 5 hours and 1 μl of the buffer solution containing biological material into the first microfluidic chamber, and the effluent media may be removed through the external outlet for analysis.

Optionally, the microfluidic cell culture device comprises a guiding element (or tab) for easy handling, placement and removal of the microfluidic cell culture device from the mufti-well cell culture plate. Optionally, the microfluidic cell culture device comprises a microbial barrier preventing the growth of microbial and/or viral contamination during growth and storage of cells.

The present disclosure also relates to the method as described above. Various embodiments and variants disclosed above, with respect to the aforementioned first aspect, apply mutatis mutandis to the method.

Optionally, the cell cultivation is a three-dimensional cell cultivation. Optionally, the three-dimensional cell cultivation is a nervous system-mimicking cell cultivation.

Optionally, the method comprises arranging the flexible non-porous membrane having a plunger area comprising one or more features to face the non-planar surface.

Optionally, the non-planar surface has one or more pits.

Optionally, the one or more features correspond to the one or more pits.

Optionally, the one or more features are perpendicular to the correspondingly arranged one or more pits.

Optionally, the one or more features are complimentary to the correspondingly arranged one or more pits.

Optionally, the method comprises arranging the one or more microfluidic channels to pass through the flexible non-porous membrane.

Optionally, the method comprises providing the biological material to the first microfluidic chamber via the one or more microchannels passing through the flexible non-porous membrane, and wherein the biological material is received into one or more pits of the non-planar surface, opposite to the flexible non-porous membrane.

Optionally, the method comprises applying a pressure to the biological material by the flexible non-porous membrane.

Optionally, debris are perfused through the one or more microfluidic channels passing through the flexible non-porous membrane.

Optionally, the method further comprises applying a chemical agent or a biological agent onto the biological material to modify at least one of: a growth and regeneration, a differentiation, a motility, a division, an adhesion, a secretion, a death, a genotype, a phenotype, a metabolism.

Optionally, the flexible non-porous membrane is depressed by applying a pressure to the second microfluidic chamber prior to adding the biological material, and wherein biological material is cultured for a predefined time after until the flexible non-porous membrane is reverted back to a neutral state thereof by reducing the pressure in the second microfluidic chamber.

The present disclosure also relates to the method of manufacturing the flexible non-porous membrane as described above. Various embodiments and variants disclosed above, with respect to the aforementioned first aspect, apply mutatis mutandis to the method of manufacturing the flexible non-porous membrane.

The method of manufacturing a flexible non-porous membrane with one or more microfluidic channels passing therethrough.

### DETAILED DESCRIPTION OF THE DRAWINGS

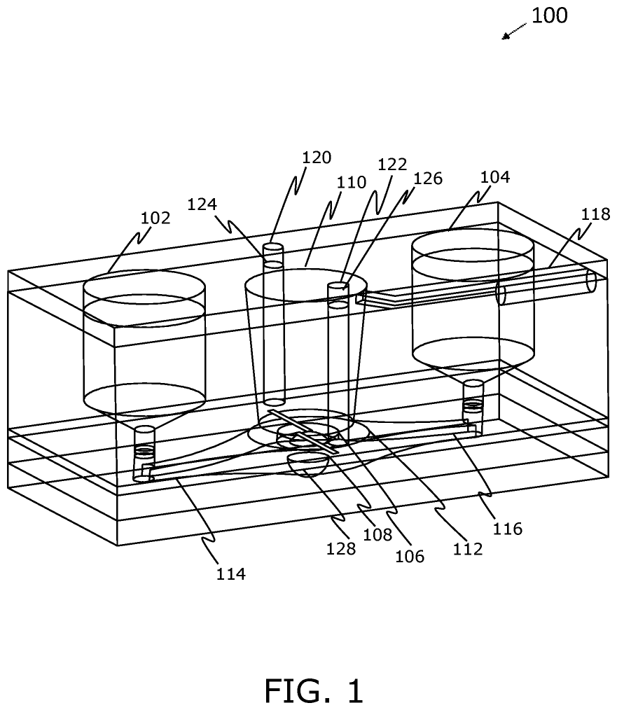

Referring to FIG. 1, there is shown a perspective view of a microfluidic cell culture device 100 for cell cultivation, in accordance with an embodiment of the present disclosure. The microfluidic cell culture device 100 comprises two or more media reservoirs, such as media reservoirs 102 and 104; a first microfluidic chamber 106 having a non-planar surface 108; a second microfluidic chamber 110 being a pressure chamber, a flexible non-porous membrane 112 that separates the first microfluidic chamber 106 and the second microfluidic chamber 110, wherein the flexible non-porous membrane 112 being opposite to the non-planar surface 108 of the first microfluidic chamber 106; and one or more microfluidic channels, such as microfluidic channels 114 and 116 connecting the first microfluidic chamber 106 to the two or more media reservoirs 102 and 104. Moreover, the second microfluidic chamber 110 comprises a pressure inlet 118 coupled to a pressure actuator (not shown). Furthermore, an outer side of the microfluidic cell culture device 100 has one or more outlets, such as outlets 120 and 122, of the one or more microfluidic channel 124 and 126, running inside the flexible non-porous membrane 112 and up vertically parallel to the second microfluidic chamber 110. Moreover, the first microfluidic chamber 106 is connected to the one or more outlets, such as outlets 120 and 122, through the one or more microfluidic channels, such as microfluidic channels 124 and 126, running inside the flexible non-porous membrane 112 and up vertically parallel to the second microfluidic chamber 110.

Moreover, the non-planar surface 108 of the first microfluidic chamber 106 comprises one or more pits, such as pit 128.

It will be appreciated that shown is a single unit of the microfluidic cell culture device 100, and a plurality of such units can be combined to form a high-throughput well plate format chips or arrays of units, where tissue wound and healing models can be performed (and analysed) in multiples. Moreover, such chips or arrays of units allows for automation as well as robotic handling. In this regard, the membrane actuation channels can be combined to form larger units to actuate multiple membranes at once with a single pressure unit.

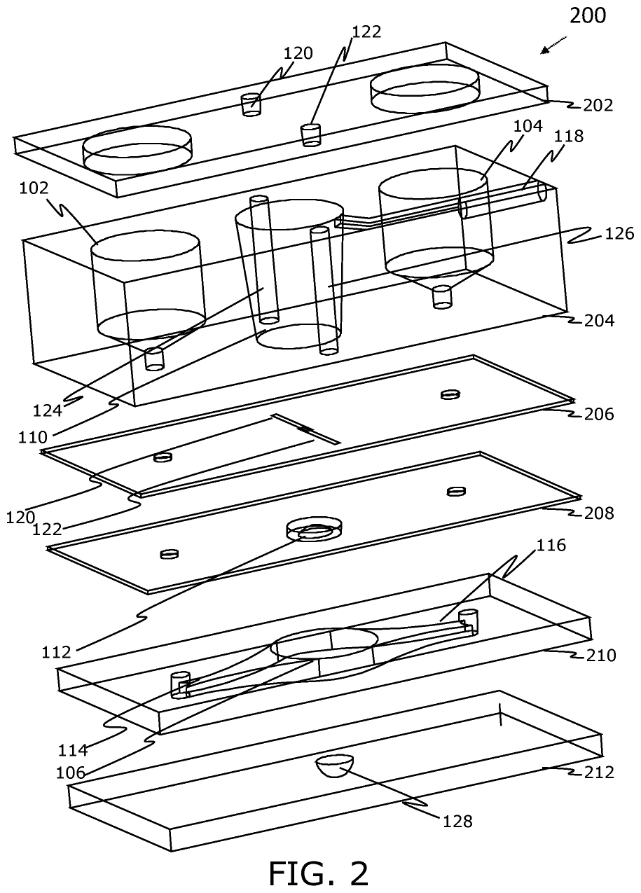

Referring to FIG. 2, there is shown an expanded view 200 of a microfluidic cell culture device 100 for cell cultivation, in accordance with an embodiment of the present disclosure. As shown, the multiple-layer architecture of the microfluidic cell culture device has six layers, i.e., a first layer 202, a second layer 204, a third layer 206, a fourth layer 208, a fifth layer 210 and a sixth layer 212, each having a predefined first, second, third, fourth, fifth, and sixth thickness, respectively. The first layer 202 of the microfluidic cell culture device 100 is the top layer that comprises openings of the two or more reservoirs 102, 104, and openings of the one or more outlets 120, 122 of the one or more microfluidic channels 124, 126. It will be appreciated that the one or more microfluidic channels 124, 126 runs vertically between the one or more outlets 120, 122 and the first microfluidic chamber 106 and through the flexible non-porous membrane 112, and the one or more microfluidic channels 114, 116 itself runs horizontally between the two or more reservoirs 102, 104 and the first microfluidic chamber 106. The second layer 204 of the microfluidic cell culture device 100 comprises the two or more reservoirs 102, 104, and the second microfluidic chamber 110 comprising a pressure inlet 118 coupled to the pressure actuator and segments of the one or more outlets 120, 122 of the one or more microfluidic channels 124, 126 passing therethrough. The third layer 206 and the fourth layer 208 of the microfluidic cell culture device 100 comprise the flexible non-porous membrane 112 comprising the plunger area (not shown) having one or more features (not shown) and segments of the one or more microfluidic channels 124, 126, running vertically between the one or more outlets 120, 122 in the first layer 202 and the first microfluidic chamber 106 in the fifth layer 210. The fifth layer 210 of the microfluidic cell culture device 100 comprises the first microfluidic chamber 106 and the one or more microfluidic channels 114, 116. Moreover, the fifth layer 210 holds the one or more microfluidic channels 114, 116 integrated into the microfluidic cell culture device 100. The sixth layer 212 of the microfluidic cell culture device 100 comprises the one or more pits, such as pit 128 in the first microfluidic chamber 106. As shown, being complementary to the one or more pits, such as pit 128 in the first microfluidic chamber 106, the plunger area is a raised cylinder with a concave indentation.

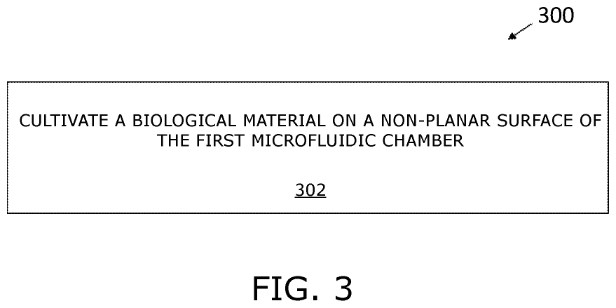

Referring to FIG. 3, there is shown a flowchart 300 of steps of a method of cell cultivation, in accordance with an embodiment of the present disclosure. At a step 302, a biological material is cultivated on a non-planar surface of the first microfluidic chamber.

The step 302 is only illustrative and other alternatives can also be provided where one or more steps are added without departing from the scope of the claims herein.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

1. A microfluidic cell culture device for cell cultivation, the microfluidic cell culture device comprising:
two or more media reservoirs;
a first microfluidic chamber having a non-planar surface;
a second microfluidic chamber being a pressure chamber,
a flexible non-porous membrane that separates the first microfluidic chamber and the second microfluidic chamber, wherein the flexible non-porous membrane being opposite to the non-planar surface of the first microfluidic chamber; and
one or more microfluidic channels connecting the first microfluidic chamber to the two or more media reservoirs.

2. The microfluidic cell culture device according to claim 1, wherein the flexible non-porous membrane has a plunger area.

3. (canceled)

4. The microfluidic cell culture device according to claim 2, wherein the plunger area comprises one or more features arranged to face the non-planar surface.

5. The microfluidic cell culture device according to claim 4, wherein the one or more features are of a cross-section.

6. (canceled)

7. The microfluidic cell culture device according to claim 1, wherein the non-planar surface has one or more pits.

8. The microfluidic cell culture device according to claim 7, wherein the one or more features correspond to the one or more pits.

9. The microfluidic cell culture device according to claim 8, wherein the one or more features are perpendicular to the correspondingly arranged one or more pits.

10. The microfluidic cell culture device according to claim 8, wherein the one or more features are complementary to the correspondingly arranged one or more pits.

11. The microfluidic cell culture device according to claim 1, wherein the one or more microfluidic channels pass through the flexible non-porous membrane.

12. The microfluidic cell culture device according to claim 1, wherein the one or more microfluidic channels in the flexible membrane have one or more outlets.

13. The microfluidic cell culture device according to claim 12, wherein each of the one or more outlets has a first end and a second end, and wherein the first end opens in to the first microfluidic chamber, and the second end opens in to at least one of:
a side corresponding to the top end of the second microfluidic chamber;
one or more biological material reservoirs (102, 104); and
an external outlet,
such that the one or more microfluidic channels connect the first microfluidic chamber to the one or more outlets.

14.-23. (canceled)

24. A method of cell cultivation using a microfluidic cell culture device of claim 1, the microfluidic cell culture device comprising:
two or more media reservoirs;
a first microfluidic chamber having a non-planar surface;
a second microfluidic chamber being a pressure chamber,
a flexible non-porous membrane that separates the first microfluidic chamber and the second microfluidic chamber, wherein the flexible non-porous membrane being opposite to the non-planar surface of the first microfluidic chamber; and
one or more microfluidic channels connecting the first microfluidic chamber to the two or more media reservoirs,
wherein the method comprises cultivating a biological material on a non-planar surface of the first microfluidic chamber.

25. The method according to claim 24, wherein the cell cultivation is a three-dimensional cell cultivation.

26. (canceled)

27. The method according to claim 24, wherein the method comprises arranging the flexible non-porous membrane having a plunger area comprising one or more features to face the non-planar surface.

28. The method according to claim 27, wherein the non-planar surface has one or more pits, wherein the one or more pits are arranged opposite to the flexible non-porous membrane and corresponding to the one or more features of the plunger area of the flexible non-porous membrane.

29. The method according to claim 24, wherein the method comprises arranging one or more microfluidic channels to pass through the flexible non-porous membrane.

30. The method according to claim 24, wherein
the method comprises providing the biological material to the first microfluidic chamber via the one or more microchannels passing through the flexible non-porous membrane, and wherein the biological material is received into one or more pits of the non-planar surface, opposite to the flexible non-porous membrane.

31. The method according to claim 24, wherein the method comprises applying a pressure to the biological material by the flexible non-porous membrane.

32. (canceled)

33. (canceled)

34. The method according to claim 24, wherein the flexible non-porous membrane is depressed by applying a pressure to the second microfluidic chamber prior to adding the biological material, and wherein biological material is cultured for a predefined time after until the flexible non-porous membrane is reverted back to a neutral state thereof by reducing the pressure in the second microfluidic chamber.

35. A method of manufacturing a flexible non-porous membrane with one or more microfluidic channels passing therethrough.
